(19) United States
(12) Patent Application Publication
(10) Pub. No.: US 2024/0006061 A1
(43) Pub. Date: Jan. 4, 2024

(54) DATA CURRENCY SYSTEM FOR DIGITAL HUMAN HEALTHCARE AND MEDICAL DATA EXCHANGE, ANALYTICS, AND APPLICATIONS

(71) Applicants: Rutgers, The State University of New Jersey, New Brunswick, NJ (US); Digital Health China Technologies, Co., Ltd., Beijing (CN)

(72) Inventors: Jing Jin, Basking Ridge, NJ (US); Wenzhao Shi, Beijing (CN); Anjiang Chen, Piscataway, NJ (US); Juan Xu, Beijing (CN); Yi Ge, Piscataway, NJ (US); Zheng Xu, Beijing (CN)

(73) Assignees: Rutgers, The State University of New Jersey, New Brunswick, NJ (US); Digital Health China Technologies, Co., Ltd., Beijing (CN)

(21) Appl. No.: 18/345,315

(22) Filed: Jun. 30, 2023

(30) Foreign Application Priority Data
Jul. 1, 2022 (CN) 202210768911.3

(51) Int. Cl. G16H 40/20; G16H 50/50; G16H 50/20; G06Q 30/0201; G06Q 20/38; G06Q 40/04; G06N 20/00

(52) U.S. Cl. CPC G16H 40/20; G16H 50/50; G16H 50/20; G06Q 30/0206; G06Q 20/389; G06Q 20/3827; G06Q 20/3829; G06Q 40/04; G06N 20/00

(57) ABSTRACT

This patent discloses a medical service platform, method, and apparatus based on a human digital twin model that comprises a a data currency valuation subsystem that provides dynamic quotes of a digital currency based on values of corresponding data in the digital human digital data currency system.

### CROSS-REFERENCE TO RELATED APPLICATIONS

This application claims priority to Chinese Patent Application No. 202210768911.3, filed Jul. 1, 2022. The foregoing application is incorporated by reference herein in its entirety.

### FIELD

Embodiments of this disclosure relate to the field of medical data production, exchange, and valuation, and to medical data, service, and financial platform, method, and apparatus for human digital twin platform.

### BACKGROUND

With the advent of the big data era, medical and health have become important fields of big data applications. Medical and health big data can be applied to many aspects, such as auxiliary diagnosis of diseases, determination of treatment plans, prediction of epidemics, analysis of drug side effects, and medical clinical research. However, the current data systems for the collection, application, analysis, and exchange of medical data are not yet perfect, so a multifunctional data system that integrates the collection, application, analysis, and exchange of medical data is needed.

### SUMMARY OF THE INVENTION

The present disclosure provides a data system and method for serving medical data exchange, analysis, and application to address the problem discussed above.

In one aspect, this disclosure provides a medical and healthcare service platform, wherein the medical and healthcare service platform is supported by a digital data currency system and provides medical and healthcare data processing, analyzing, and predicting based on a digital human system by integrating participating parties comprising individual persons, researchers, healthcare providers, and regulatory and public sectors.

In some embodiments, the medical and healthcare service platform comprises:

a digital human replica system that constructs a digital human replica to provide virtual representation, modeling, and visualization services based on present and past medical and health data of physical persons;

a digital human simuli system that constructs a digital human simuli to provide virtual simulation and modeling of future health and physiological evolution of a physical person based on the present and past medical and health data;

a digital human agent system that represents virtual medical and health service professionals with specialties and functions, wherein the virtual medical and healthcare service professionals are formed based on professional knowledge and capabilities, specialties, and experiences of physical medical and healthcare professionals and characteristics and specialties of non-medical and healthcare professionals or practitioners; and

a digital human data acquisition system that collects biometric identification and medical-related data,

wherein the digital data currency system awards data sharing and contributions in a full ecosystem of data generation comprising data processing, data cleaning and denoising, data encryption and anonymization, data labeling and calibration, data analytics, and data is contributions related to medical and healthcare services; and services provided by medical and healthcare professionals from clinical practices, drug companies from laboratories or clinical trial data, and academic researchers from research work,

wherein the digital human replica system receives input data from the human data acquisition system for both a target physical person and other persons with similar biomedical, social-demographical, occupational, and lifestyle characteristics for building, calibrating, and customizing the digital human simuli system for the target physical person to simulate the growing and aging, disease events, injury events, and their reactions to medicines and treatment plans,

wherein the digital human agent system creates a special digital human replica of actions, treatment plans, and decision makings of medical and healthcare professionals, the digital human agent system configured to execute simulated medical, care, and health services as an intervention based on simulation in a digital human simuli model of the digital human simuli system,

wherein the digital human stimuli and the digital human agent system are integrated to perform model optimization to select and determine an optimal treatment or support plan to achieve an optimal health and medical outcome,

wherein the digital human digital data currency system awards digital currency for data contribution by the participating parties who interface with the digital human data acquisition system and wherein data contribution results in improvement of performance of the digital human simuli system and the digital human agent system, and

wherein the digital human digital data currency system comprises a data currency valuation subsystem that provides dynamic quotes of a digital currency based on values of corresponding data in the digital human digital data currency system.

In some embodiments, the digital human digital data currency system further comprises: i) a medical data input interface for data uploading and transferring from hospitals, individuals, public agencies, families, and communities, ii) a data feature-based value evaluation subsystem that defines data currency valuation based on timeliness, acquisition or processing cost of data, frequency of data usage, new medical health advances including new artificial intelligence (AI) models, drugs, treatment, and medical devices, public and is community health value increase, and data quality and security related dynamic features, iii) a data quality assurance and quality control subsystem that ensures consistency and standards of data products, iv) a data currency volume and ownership assignment subsystem that generates new data currency based on AI model performance improvement and distributes generated currency based on contributions of all participants of providing, processing, cleaning, and modeling data, v) a data acquisition, processing, and labeling contribution assessment subsystem that conducts production of cleaned, anonymized, labeled datasets for healthcare AI models and applications, vi) a hybrid federated and machine learning based contribution assessment system to assess contributions of data contributors with different direct data sharing or indirect AI model weights sharing for improving healthcare AI model and applications.

In some embodiments, the medical data input interface is configured to collect medical related data and comprises: (a) a hospital interface for uploading and transferring of medical records from doctors and hospital data center, (b) a personal interface for individual to upload real-time or historical wearable healthcare device data, medical exam results, and direct text—or audio-based inputs, (c) a public agency interface to collect anonymous data collected from health checkpoint and population, and (d) a family and community interface for uploading case-by-case descriptions and audio and video data.

In some embodiments, the data currency valuation subsystem uses data features for determining initial data value evaluation, wherein the data features comprise data types or categories for application; data stage of medicine trials; infectious disease severity designations; individual disease stage designation; data sources; social demographics; data fields; data coverage; data volume; or data resolution.

In some embodiments, the data currency valuation subsystem determines monetary tokenized valuation for healthcare-related data, and wherein: a) value appreciation is determined by one or more of the following factors: timeliness of new or real-time data, wherein value of the data positively correlates with timeliness of the data; high-frequency to effective data usage; a new drug, device, or treatment created or discovered; a public health value added through contributions to new community and public health solutions to detect and control spreading and outbreak an infectious disease; data processing, computing, and production cost in a full data production process; cyber security improvement to enhance data security and improved counterfeit countermeasures; data-assisted AI model and application is performance improvement such as accuracy and coverage; and data currency trading related price increase; or b) value deprecation is determined by one or more of the following factors: increased number of repeated or outdated datasets that do not provide new or useful information for healthcare model and application improvement; increased number of bad records in data production comprising missing data, fake data, or biased data; leaking, counterfeit, and illegal usage of raw or confidential datasets that lead to legal, technological, societal, and sale challenges of datasets in practices of healthcare model and application; reduced effective data usage by models and applications; and data currency trading related price decrease.

In some embodiments, the data currency valuation subsystem determines value evaluation of medical-related data based on one or more of data features comprising: a) standardization module of data production from raw data formatting, data processing procedure, analytic and labeling tools and functionality specifications, and data performance and quality assurance and control; b) data quality assessment metrics comprising: comparison to validation or diagnostics datasets; detection or diagnostic accuracies; recovery rates and quality assessment; death, incidents, severe damage, injuries, or paralyzation rate; c) erroneous and false data detection and correction methods comprising: sensor error and malfunctions; human input error or vagueness; or text and handwriting recognition error; and d) multi-dataset cross-validation mechanisms comprising: spatial-temporal matching; correlation matching; or pattern matching between similar population groups.

In some embodiments, the data quality assurance and quality control, and reporting subsystem comprises a periodic data quality reporting system that reports one or more of:
a) cost of data production and maintenance comprising one or more of: data generation and/or acquisition cost, data cleaning and labeling cost, data analytic cost, data storage cost, data application cost, and security, certification, or oversight cost;
b) number of applications and application revenue and monetization results in a reporting period comprising one or more of data application subscription and membership revenues; data application service revenues; data currency trading revenues; monetized values of derived products such as new drugs, treatment methods, derived data applications; and money savings for families, agencies, and communities compared with prior applications or methods; and
c) a data product quality metric system comprising one or more of: i) output versus input instance ratios defined as a ratio between application service instances over number of input data instances in a reporting period; ii) data growth capability defined by processed, labeled, and calibration-consumed data versus newly acquired or produced data rate based on size of training data set to achieve a latest model divided by a rate of new data acquisition through applications; iii) incremental data benefit gains to keep track of different stages of data and application matureness, especially, monitoring mature datasets entering a long-tail stage where increased data consumption is needed to gain performance improvement of trained AI models and applications; iv) user growth potentials based on a growth rate of users of healthcare products or applications produced by the data, average cost per user data acquisition and production, and usage activity data based on active user engagement time and interactions; and v) data supported models, publications, and application increasing rates.

In some embodiments, the data currency volume and ownership assignment system allocates and distributes currency based on each round of optimization of parameters and gradients of the AI model, and wherein the data currency volume and ownership assignment system comprises one or more of: a) a new data currency module that is generated with every round of calibration converges triggered by new data, new model, new optimization method; b) a parameter or gradient update module to keep track of performance improvement in accuracy, coverage, and other performance metrics of the data; c) a hashing system that generate unique data currency lot ID based with creating a hash code corresponding to updated model performance metrics, parameters, or gradients; d) a data currency pooling system to record and store new data currency lots and status of a new gradient or parameter combination and corresponding performance gains; e) a blockchain-enabled global ledger to register data currency generation and pooling activities with every round of such updates; and f) a data currency assignment module that distributes new data currency lots in a new currency pool to all contributors of calibration or training, including new data contributors to raw, labeled, and ground truth data, computing resource providers for edge or cloud computing depending on AI to technology architectures, validators, data and file transmission, or cyber security measures.

In some embodiments, the data currency valuation subsystem comprises a calibration data currency volume and ownership assignment system comprising one or more of: calibration datasets oriented; large-scale parameter settings that use a tree structure to compress and tokenization; and all sets of parameters to be created in a way that follows an evolution tree; and new currency key or token with encryption essentially mapping uniqueness of structure and uniqueness of data nodes to form a unique encryption key or token.

In some embodiments, the data acquisition, processing, and labeling contribution assessment system comprises one or more of: a) historical data depreciation methods by using statistical distribution models for modeling and calibrating decay with respect to time; b) dynamic and real-time data valuation methods for determining randomness of datasets or predictability of the datasets from historical data; and c) rare and high-value data valuation metrics comprising targeted data types, number of rare, high-value data occurrences, or frequencies.

In some embodiments, the data currency valuation subsystem comprises a federated and machine learning contribution assessment system comprising one or more of:
i) direct machine learning or deep learning assessment where currency volume is allocated based on performance improvement of models and applications through centralized machine learning model calibration with direct raw data and data labels produced at a data center;
ii) a federated learning assessment module that assesses currency volume only based on performance improvement and parameter, weight or gradient updates from federated learning process where raw data are not shared with a data center;
iii) machine learning and federated learning contributions comprising: dataset contributions with respect to increment in gradients or parameters changes or dataset contributions in improvement of data precision, accuracy, or coverage;
iv) machine-learning- and federated-learning-based data currency allocation based on one or more of: a) an allocation submodule based on completeness and development of a model where allocation will increase with higher average change rates of gradient or parameters in an AI model as a result of a training process, and will decrease if average change in parameters or gradients of the model caused by the dataset used is not significant; b) an allocation submodule based on processed or used percentage of input and label data; c) an allocation submodule based on improvement in model accuracy where allocation increases with larger accuracy improvement beyond a pre-defined no-change threshold specified by a data product and decreases with smaller accuracy improvement in trained models; and d) an allocation rule integration module that determines final allocation based on a combined assessment of the is submodules set forth in a)-c) based on characteristics of data products.

In some embodiments, the data currency valuation subsystem comprises a data currency exchange platform comprising one or more of the following functionalities: a) data application that produces user groups registration and management including one or more of: data contributors who upload raw data; data collectors, surveyors, and buyers; data processors and labelers; data brokers and resellers; data application developers or operators; data application end users; data analysts that produce insights towards developing corresponding models and applications; and data users through digital currency traders and transactions; b) a data currency exchange cryptocurrency transaction process function comprising one or more of: initiate transaction process; secure transaction function; point-to-point transaction; transaction group-to-group; validate process; certify process; recording process; and transferring; c) smart multi-party contracting among data contributors; d) digital currency transactions logging and management system; e) currency token circulation within an exchange platform; f) toke circulation and exchange with data currency from other exchange platforms or other currency or cryptocurrencies; and g) security and user protection modules.

In some embodiments, the data currency exchange platform has a data currency exchange function for internal platform circulation, wherein within one data currency platform, data currency from different data currencies can be tradeable to be used to purchase or exchange data products under other data currencies, wherein different types of data products are purchased, exchanged, and acquired, using the same data currency, wherein for external exchange with other currencies, the data currency platform comprises an exchange module for data currencies from other data currency exchange systems, an exchange module with world currency with a specific exchange rate, or an exchange module with other cryptocurrencies, and wherein exchange rates are determined by one or more of characteristics: an exchange rate system between different data currency types or similar data products under different data currency; exchange with world currency once money values are determined through initial valuation process; all data products have value, but not all data products have transaction value;

some data products are protected and never tradeable; and a need to define a method for dividing data products.

In some embodiments, the data currency valuation subsystem performs a data currency global certification process that allows distributed systems to work together and stay secure for is generating data currency, wherein the data currency global certification process comprises one or more of: a) Proof-of-Work (PoW) consensus mechanisms based on hours or result instances processed, analyzed, labeled, or computed by different data contributors; and b) Proof-of-Stake (PoS) consensus mechanisms based on data currency generation and allocation module outcomes.

In some embodiments, the data currency operation and transaction subsystem comprises a data currency development framework for building, distribution, issuance, regular disclosure, property rights protection, the data currency development framework comprising one or more of: a) encryption and cyber security modules that comprises central and individual data currency information security modules; anti-theft and anti-piracy management for data sources; and digital oversight system to implement regulatory requirements by government agencies; b) data currency blockchain platform to provide global verifiable ledgers to document data currency transactions and activities; c) data currency token or coin development modules; d) data currency smart testing process comprising: deploying tokens on a testnet; deploying smart contracts; or verifying source code; e) data currency smart contracts and wallet modules for individual data currency keeping and exchange, which performs one or more operations of: (i) transaction management of smart contracts; (ii) initiating, approving, and verifying transfers or trades; (iii) satisfying factor authentications and maintenance security keys; (iv) enforcing trading and transaction rules and regulatory requirements; (v) initial price and smart contract offering; (vi) monitoring and maintaining timing records of transactions; and f) data currency intellectual property protection and disclosure systems.

In some embodiments, the data currency development framework comprises a data currency token or coins development platform comprising one or more of: a) data currency tokens standards and templates; b) data currency digital wallet; c) smart contract interface; d) transfer function interface; e) record keeping interface to document name, symbol, and decimal of a token; f) initial total supply management interface; g) data and application product documentation interface; h) AI model structure, parameter, and performance metrics to documentation interface; i) programming interface and coding environment; j) application development interfaces to interact with other digital currencies; and k) application development interfaces to interact with trading and transaction systems.

In some embodiments, the data currency development framework comprises a data currency smart distribution process comprising one or more of: initial coin offering (ICO) or is holding a crowd sale; build and/or maintain user community; white papers, laws, and regulations; and official release, transaction, operation and maintenance.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 is a schematic diagram of the digital human medical and healthcare data currency system.

FIG. 2 is a schematic diagram of the data currency valuation system.

FIG. 3 is a schematic diagram of the data currency volume allocation system.

FIG. 4 is a schematic diagram of the data currency contribution determination system.

FIG. 5 is a schematic structural diagram of data currency exchange platform.

FIG. 6 is a schematic structural diagram of data currency operation and transaction system.

### DETAILED DESCRIPTION

This disclosure provides a data currency system that serves medical data exchanges, analyses, and applications. The system comprises a data currency valuation subsystem that provides dynamic quotes of a digital currency based on values of corresponding data in the digital human digital data currency system.

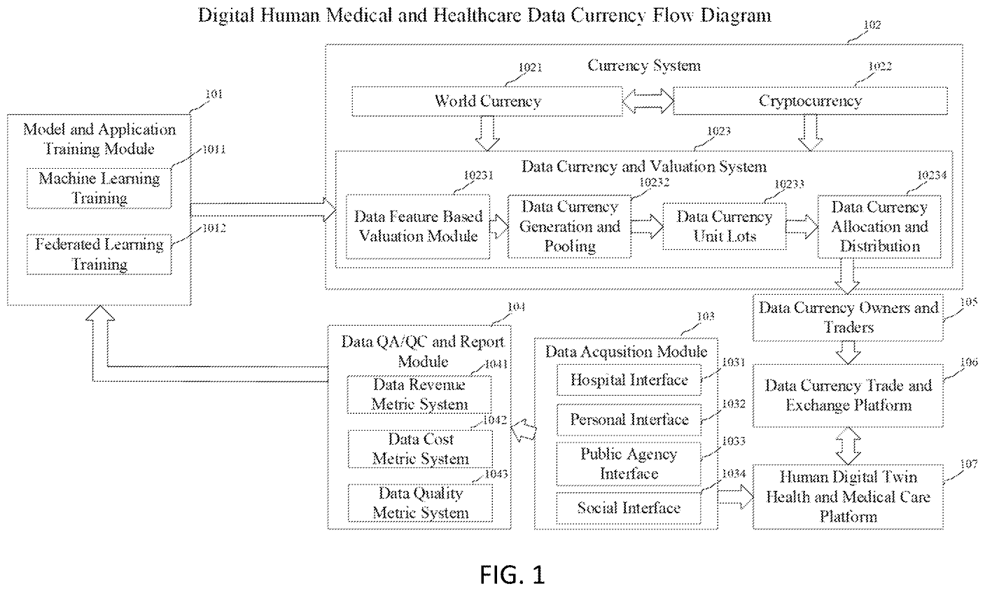

As shown in FIG. 1, the data currency system starts from the healthcare and medical data acquired through interfaces developed for hospitals 1031, individuals themselves 1032, public agencies (1033, and families and communities input interfaces 1034. Hospitals 1031 can upload anonymous medical data and records and/or match medical data or records with is data currency platform participants or contributors with data disclosure consent. Individuals 1032 can use personal interfaces to upload data, which include real-time or periodic synchronization interfaces for wearable devices, web interfaces for reporting symptoms, medical or family history, exercise or treatment routines, and medical events, manual uploading of medical records, and/or granting access to one's health and medical records. Public agencies 1033 such as public health organizations, emergency response agencies, industrial associations, and organizations can also link their public data sources or data feeds and upload aggregated population and health data according to agency regulations, policies, and guidelines. Families and communities 1034 can also help contribute data for particular patients or healthy persons who opted into the human digital twin platforms by providing their descriptions, observations, medical exam or data records, and support history for the patients or persons of interest.

The data quality assurance/quality control (QA/QC) module 104 is used to monitor and periodically report on the data quality and operational quality. The module includes the revenue report interface, used to monitor the data quality based on a quality metric system 1043 that covers different aspects of accuracy, popularity, sustainability, effectiveness, coverage, security, and others. The revenue metric system 1041 is used to track all potential revenue streams, and the cost metric system 1042 is used to account for all the related costs of acquiring, creating, processing, modeling, and using the data products.

The QA/QC and reporting module will interact with the model and application training module 101 that can operate in two modes, the machine learning mode and the federated learning model. In machine learning training 1011, all the raw data are sent to a centralized server at the digital human medical and healthcare data platform and artificial intelligence models are calibrated by directly inputting the raw data and labels. Federated learning training 1012 is used when the raw data may have issues to be sent directly the digital human data platforms due to privacy and security concerns of different participating entities. In federated learning training 1012, the raw data are kept behind the firewall of participating entities, and the training of the AI models is conducted at servers deployed inside the firewall of those entities, and only the trained parameters and weights are provided to digital human data platforms where those weights and parameters will be integrated with other training results. At the core of the currency system 102 is the data currency and valuation system 1023 will execute is the main data currency generation and pooling 10232, feature-based valuation 10231, the hosting of main data currency unit lots 10233, the allocation and distribution of the data currency 10234 to the data contributors. Once received, those data contributors become the owners and potential traders of the data currency. The data currency trade and exchange platform 106 is then established to handle the currency trading and interact with the applications and models in the human digital twin platform 107.

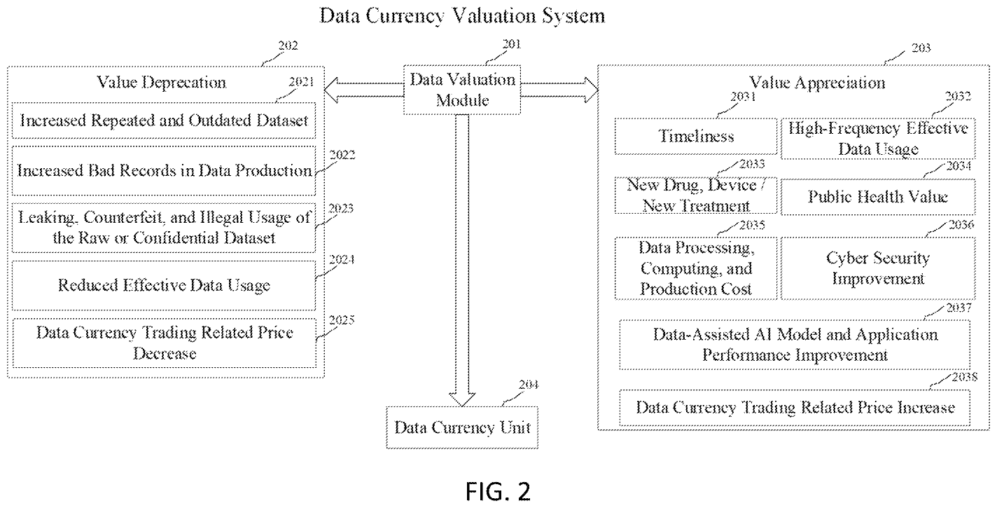

Referring now to FIG. 2, the data currency valuation system uses the data valuation module 201 to determine value deprecation 202 and value appreciation 203 and affect data currency unit 204. Increased repeated and outdated dataset 2021, increased bad records in data production 2022, leaking, counterfeit, and illegal usage of the raw or confidential dataset 2023, reduced effective data usage 2024, and data currency trading related price decrease 2025 result in the value deprecation 202. The factors that will cause value appreciation 203 include timeliness 2031, high-frequency effective data usage 2032, new drug, device/new treatment 2033, public health value 2034, data processing, computing, and production cost 2035, cyber security improvement 2036, data assisted AI model and application performance improvement 2037, and data currency trading related price increase 2038.

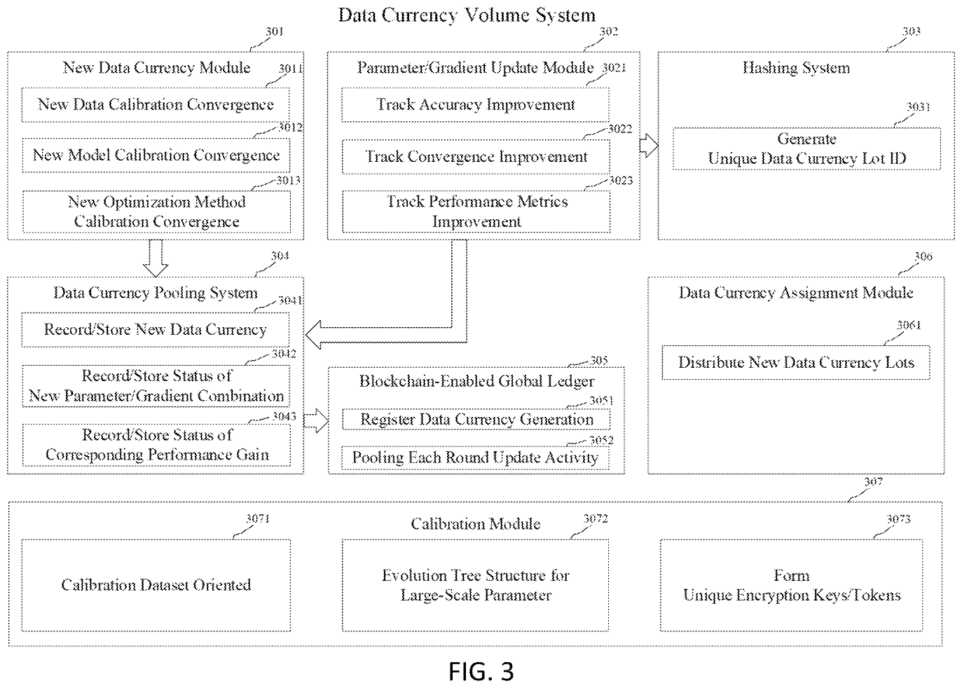

As shown in FIG. 3, the data currency volume system has a new data currency module 301, a parameter/gradient update module 302, a hashing system 303, a data currency pooling system 304, a blockchain-enabled global ledger system 305, and a data currency assignment module 306. The new data currency module 301 can be triggered every time a new data currency is generated. The triggering principles include new data calibration convergence 3011, mew model calibration convergence 3012, new optimization method calibration convergence 3013. The parameter/gradient update module 302 is to track the improvement of accuracy 3021, convergence 3022, and performance metrics 3023, which are the principle of the data currency allocation and distribution.

The hashing system 303 is to generate unique data currency lot ID 3031 based on the parameter/gradient update module 302. Therefore, each improvement activity will have an unique hashing encryption code to record. The new data currency pooling system 304 is to record and store new data currency generated in new data currency module 301 and parameter/gradient update module 302. Namely, it can record/store new data currency 3041, record/store status of new parameter/gradient combination 3042 and record/store status of corresponding performance combination 3043.

The blockchain-enabled global ledger system 305 is to register the data currency generation 3051 and pooling each round update activity 3052 from new data currency pooling system 304. The data currency assignment module 306 is to distribute new data currency lots 3061 to all the contributors. In addition, the data currency volume system also has a calibration module 307. The calibration module is calibration dataset oriented 3071, an evolution tree structure for large-scale parameter 3072 to compress and tokenization, and to form unique encryption keys/tokens 3073 to map the uniqueness of the structure and the uniqueness of data nodes.

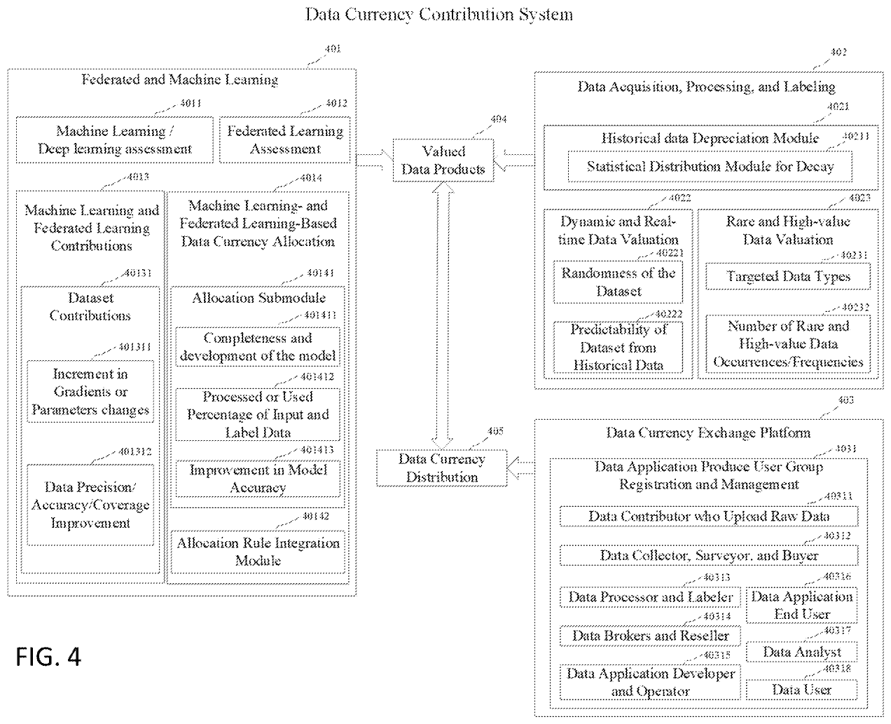

As shown in FIG. 4, the data currency contribution system uses valued data products 404 to determine the data currency distribution 405. The valued data products 404 includes a subsystem for federated and machine learning 401 and a subsystem for the data acquisition, processing, and labeling 402. The subsystem for federated and machine learning 401 takes into account the machine learning/deep learning assessment 4011, federated learning assessment 4012, machine learning and federated learning contributions 4013, and machine learning- and federated learning-based data currency allocation 4014. The machine learning and federated learning contributions 4013 considers dataset contributions 40131 including increment in gradients or parameters changes 401311 and data precision/accuracy/coverage improvement 401312. The machine learning- and federated learning-based data currency allocation 4014 contains two submodules, allocation submodule 40141 and allocation rule integration module 40142. The allocation submodule 40141 considers the completeness and development of the model 401411, the processed or used percentage of input and label data 401412, and the improvement in model accuracy 401413.

The data acquisition, processing, and labeling 402 contains three subsystems, historical data depreciation module 4021, dynamic and real-time data valuation 4022, and rare and high-value data valuation 4023. The historical data depreciation module 4021 contains a statistical distribution module for decay 40211. The dynamic and real-time data valuation contains randomness of the dataset 40221, and predictability of dataset from historical data 40222. The rare and high-value data valuation 4023 contains targeted data types 40231 and number of rare and high-value data occurrences/frequencies 40232. The data currency distribution 405 uses a data currency exchange platform 403 for data application produce user group registration and management 4031, which includes data contributors who upload raw data 40311, data collector, surveyor, and buyer 40312, data processor and labeler 40313, data brokers and is reseller 40314, data application developer and operator 40315, data application end user 40316, data analyst 40317, and data user 40318.

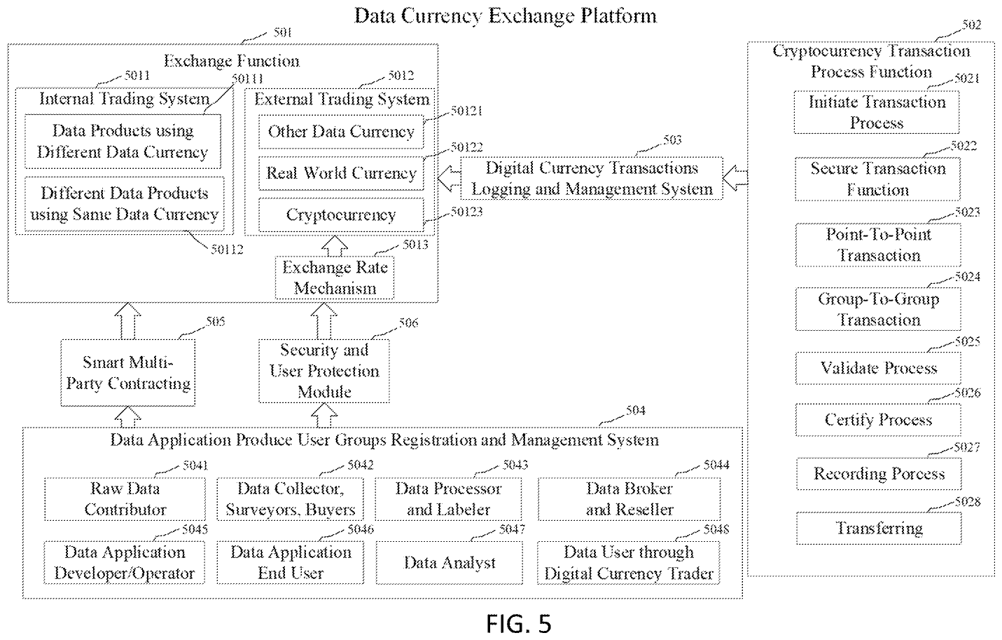

As shown in FIG. 5, the data currency exchange platform starting from an exchange function 501, a cryptocurrency transaction process function 502, and a digital currency transactions logging and management system 503. The exchange function 501 has two main subsystems: internal trading system 5011 and external trading system 5012. The internal trading system 5011 serves for two types of internal circulations: Data products using different data currency 50111 and different data products using same data currency 50112. The external trading system 5012 serves for exchange with other currencies, including other data currency 50121, real world currency 50122 and cryptocurrency 50123. The exchange rate mechanism 5013 in real time is served for fair trading between different currencies.

The cryptocurrency transaction process function 502 comprises one or more of: Initiate transaction process 5021, Secure transaction function 5022, Point-to-point transaction 5023, Group-to-group transaction 5024, Validate process 5025, Certify process 5026, Recording process 5027, and transferring 5028. The cryptocurrency transaction process function will interact with exchange function through digital currency transactions logging and management system 503. The data application produces user groups registration and management system 504 contains one or more of user groups: Raw data contributor 5041, Data collector, surveyors, buyers 5042, Data processor and labeler 5043, Data broker and reseller 5044, Data application developer/operator 5045, Data application end user 5046, Data analyst 5047, and Data user through digital currency trader 5048. Between exchange function 501 and data application produce user groups registration and management system 504, there are smart multi-party contracting 505 and security and user protection module 506 to execute exchange process and ensure the security of the exchange process.

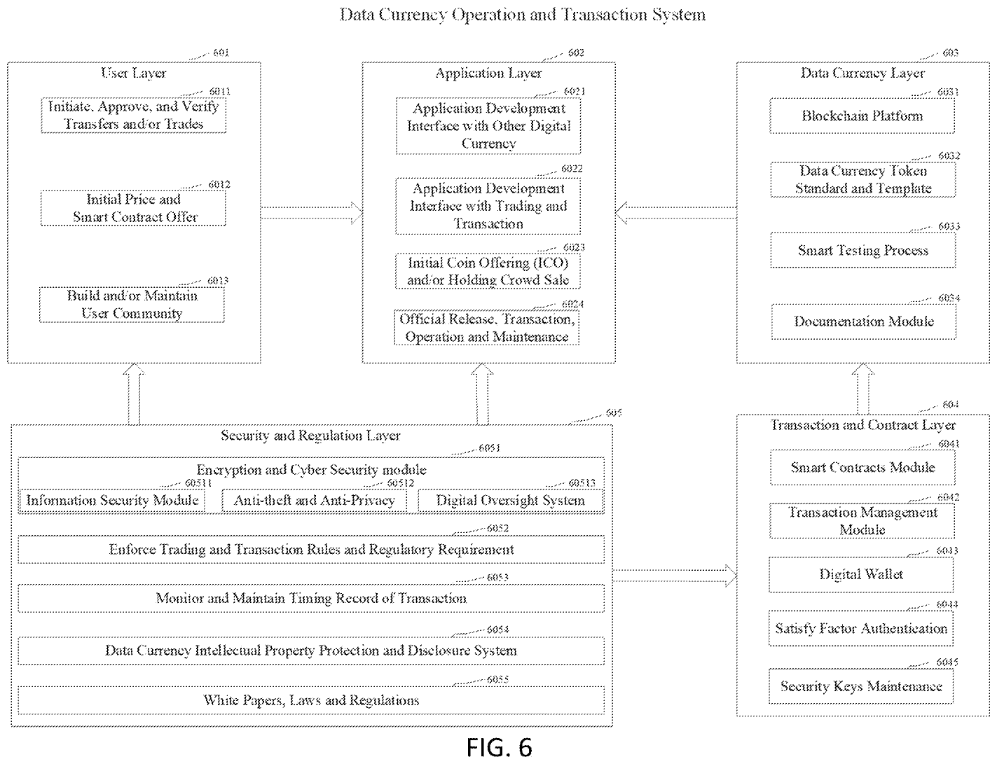

As shown in FIG. 6, the data currency operation and transaction system has five layers: user layer 601, application layer 602, data currency later 603, transaction and contract layer 604, security and regulation layer 605. The user layer 601 provide the functions for use to engage the data currency transaction system with initiate, approve and verify transfers and trades 6011, provide initial price and smart contract offer 6012 of data currency, and build and/or maintain user community 6013. The application layer 602 provides the functions to support all the applications in the data currency operation and transaction system, which comprises an application development interface with other digital currency 6021, an is application development interface with trading and transaction 6022, Initial Coin Offering (ICO) and/or holding crowd sale 6023, and provide official release, transaction, operation, and maintenance 6024 from the first time release activity to a long term operation of data currency.

The user layer 601 engage the data currency operation and transaction system through the application layer 602. The data currency layer 603 comprising of all the necessary components of data currency, including a blockchain platform 6031 to provide global verifiable ledgers, a data currency token standard and template 6032, a smart testing process 6033 to deploy tests on a testnet, deploy smart contract and verify source code, and a documentation module 6034 to document data and application products, AI model structure, parameter, performance metrics, and keep all the records. The data currency layer 603 interacts with the application layer 602 to operate and/or distribute, and/or allocate.

The transaction and contract layer 604 provide the functions for data currency operation and transaction, comprising of a smart contract module 6041, a transaction management module 6042, a digital wallet 6043 to store and collect data currency, a satisfy factor authentication 6044 and a security keys maintenance 6045. The transaction and contract layer 604 provide the operation and transaction functions for data currency layer 603. The security and regulation layer 605 comprising of security functions and provide regulation requirements for user layer 601, application layer 602, data currency layer 603, and transaction and contract layer 604.

The security and regulation layer 605 provides an encryption and cyber security module 6051, which comprises of an information security module 60511 for the central and individual data currency information security, an anti-theft and anti-piracy 60512 system for the management of data sources, and a digital oversight system 60513 to implement regulatory requirements by government agencies. The security and regulation layer 605 is used to enforce trading and transaction rules and regulatory requirement 6052 and monitor and maintain timing record of transaction 6053 for the transaction and contract layer 604. The security and regulation layer 605 provide a data currency intellectual property protection and disclosure system 5044 for the data currency layer 603. The security and regulation layer 605 provides whites papers, laws, and regulations for user layer 601 and application layer 602.

In one or more embodiments, as described in FIG. 1, the data currency system starts from the healthcare and medical data acquired through interfaces developed for hospitals (1031), individuals themselves (1032), public agencies (1033), and families and communities is input interfaces (1034). Hospitals (1031) can upload anonymous medical data and records and/or match medical data or records with data currency platform participants or contributors with data disclosure consent. Individuals (1032) can use personal interfaces to upload data, which include real-time or periodic synchronization interfaces for wearable devices, web interfaces for reporting symptoms, medical or family history, exercise or treatment routines, and medical events, manual uploading of medical records, and/or granting access to one's health and medical records. Public agencies (1033) such as public health organizations, emergency response agencies, industrial associations, and organizations can also link their public data sources or data feeds and upload aggregated population and health data according to agency regulations, policies, and guidelines. Families and communities (1034) can also help contribute data for particular patients or healthy persons who opted into the human digital twin platforms by providing their descriptions, observations, medical exam or data records, and support history for the patients or persons of interest.

The data quality assurance/quality control (QA/QC) module (104) is used to monitor and periodically report on the data quality and operational quality. The module includes the revenue report interface, used to monitor the data quality based on a quality metric system (1043) that covers different aspects of accuracy, popularity, sustainability, effectiveness, coverage, security, and others. The revenue metric system (1041) is used to track all potential revenue streams, and the cost metric system (1042) is used to account for all the related costs of acquiring, creating, processing, modeling, and using the data products.

The QA/QC and reporting module will interact with the model and application training module (101) that can operate in two modes, the machine learning mode and the federated learning model. In machine learning training (1011), all the raw data are sent to a centralized server at the digital human medical and healthcare data platform and artificial intelligence models are calibrated by directly inputting the raw data and labels. Federated learning training (1012) is used when the raw data may have issues to be sent directly the digital human data platforms due to privacy and security concerns of different participating entities. In federated learning training (1012), the raw data are kept behind the firewall of participating entities, and the training of the AI models is conducted at servers deployed inside the firewall of those entities, and only the trained parameters and weights are provided to digital human data platforms where those weights and parameters will be integrated with other training results. At the core of the currency system (102) is the data currency and valuation system (1023) will execute the main data currency generation and pooling (10232), feature-based valuation (10231), the hosting of main data currency unit lots (10233), the allocation and distribution of the data currency (10234) to the data contributors. Once received, those data contributors become the owners and potential traders of the data currency. The data currency trade and exchange platform (106) is then established to handle the currency trading and interact with the applications and models in the human digital twin platform (107).

In one or more embodiments, as described in FIG. 2, the data currency valuation system uses the data valuation module (201) to determine value deprecation (202) and value appreciation (203) and affect data currency unit (204). Increased repeated and outdated dataset (2021), increased bad records in data production (2022), leaking, counterfeit, and illegal usage of the raw or confidential dataset (2023), reduced effective data usage (2024), and data currency trading related price decrease (2025) result in the value deprecation (202). The factors that will cause value appreciation (203) include timeliness (2031), high-frequency effective data usage (2032), new drug, device/new treatment (2033), public health value (2034), data processing, computing, and production cost (2035), cyber security improvement (2036), data assisted AI model and application performance improvement (2037), and data currency trading related price increase (2038).

As shown in FIG. 3, the data currency volume system has a new data currency module (301), a parameter/gradient update module (302), a hashing system (303), a data currency pooling system (304), a blockchain-enabled global ledger system (305) and a data currency assignment module (306). The new data currency module (301) can be triggered every time a new data currency is generated. The triggering principles include new data calibration convergence (3011), mew model calibration convergence (3012), new optimization method calibration convergence (3013). The parameter/gradient update module (302) is to track the improvement of accuracy (3021), convergence (3022), and performance metrics (3023), which are the principle of the data currency allocation and distribution.

The hashing system (303) is to generate unique data currency lot ID (3031) based on the parameter/gradient update module (302). Therefore, each improvement activity will have a unique hashing encryption code to record. The new data currency pooling system (304) is to record and store new data currency generated in new data currency module (301) and parameter/gradient update module (302). Namely, it can record/store new data currency is (3041), record/store status of new parameter/gradient combination (3042) and record/store status of corresponding performance combination (3043). The blockchain-enabled global ledger system (305) is to register the data currency generation (3051) and pooling each round update activity (3052) from new data currency pooling system (304). The data currency assignment module (306) is to distribute new data currency lots (3061) to all the contributors. In addition, the data currency volume system also has a calibration module (307). The calibration module is calibration dataset oriented (3071), an evolution tree structure for large-scale parameter (3072) to compress and tokenization, and to form unique encryption keys/tokens (3073) to map the uniqueness of the structure and the uniqueness of data nodes.

In one or more embodiment, as described in FIG. 4, the data currency contribution system uses valued data products (404) to determine the data currency distribution (405). The valued data products (404) include a subsystem for federated and machine learning (401) and a subsystem for data acquisition, processing, and labeling (402). The subsystem for federated and machine learning (401) takes into account the machine learning/deep learning assessment (4011), federated learning assessment (4012), machine learning and federated learning contributions (4013), and machine learning- and federated learning-based data currency allocation (4014). The machine learning and federated learning contributions (4013) considers dataset contributions (40131) including increment in gradients or parameters changes (401311) and data precision/accuracy/coverage improvement (401312).

The machine learning- and federated learning-based data currency allocation (4014) contains two submodules, allocation submodule (40141) and allocation rule integration module (40142). The allocation submodule (40141) considers the completeness and development of the model (401411), the processed or used percentage of input and label data (401412), and the improvement in model accuracy (401413). The data acquisition, processing, and labeling (402) contains three subsystems, historical data depreciation module (4021), dynamic and real-time data valuation (4022), and rare and high-value data valuation (4023). The historical data depreciation module (4021) contains a statistical distribution module for decay (40211). The dynamic and real-time data valuation contains randomness of the dataset (40221), and predictability of dataset from historical data (40222). The rare and high-value data valuation (4023) contains targeted data types (40231) and number of rare and high-value data occurrences/frequencies (40232). The data currency distribution (405) uses a data currency exchange platform (403) for data application to produce user group registration and management (4031), which includes data contributors who upload raw data (40311), data collector, surveyor, and buyer (40312), data processor and labeler (40313), data brokers and reseller (40314), data application developer and operator (40315), data application end user (40316), data analyst (40317), and data user (40318).

As shown in FIG. 5, the data currency exchange platform starting from an exchange function (501), a cryptocurrency transaction process function (502), and a digital currency transactions logging and management system (503). The exchange function (501) has two main subsystems: internal trading system (5011) and external trading system (5012). The internal trading system (5011) serves for two types of internal circulations: Data products using different data currency (50111) and different data products using same data currency (50112). The external trading system (5012) serves for exchange with other currencies, including other data currency (50121), real world currency (50122) and cryptocurrency (50123). Wherein, the exchange rate mechanism (5013) in real time is served for fair trading between different currencies. The cryptocurrency transaction process function (502) comprising one or more of: Initiate transaction process (5021), Secure transaction function (5022), Point-to-point transaction (5023), Group-to-group transaction (5024), Validate process (5025), Certify process (5026), Recording process (5027), and transferring (5028). The cryptocurrency transaction process function will interact with exchange function through digital currency transactions logging and management system (503).

The data application produces user groups registration and management system (504) containing one or more user groups: Raw data contributor (5041), Data collector, surveyors, buyers (5042), Data processor and labeler (5043), Data broker and reseller (5044), Data application developer/operator (5045), Data application end user (5046), Data analyst (5047), and Data user through digital currency trader (5048). Between exchange function (501) and data application produce user groups registration and management system (504), there are smart multi-party contracting (505) and security and user protection module (506) to execute exchange process and ensure the security of the exchange process.

As shown in FIG. 6, the data currency operation and transaction system has five layers: user layer (601), application layer (602), data currency later (603), transaction and contract layer (604), and security and regulation layer (605). The user layer (601) provides the functions for users to engage the data currency transaction system with initiate, approve, and verify transfers and trades (6011), provide initial price and smart contract offer (6012) of data currency, and build and/or maintain user community (6013).

The application layer (602) provides the functions to support all the applications in the data currency operation and transaction system, which comprises an application development interface with other digital currency (6021), an application development interface with trading and transaction (6022), Initial Coin Offering (ICO) and/or holding crowd sale (6023), and provide official release, transaction, operation and maintenance (6024) from the first time release activity to a long term operation of data currency. The user layer (601) engages the data currency operation and transaction system through the application layer (602).

The data currency layer (603) comprising of all the necessary components of data currency, including a blockchain platform (6031) to provide global verifiable ledgers, a data currency token standard, and template (6032), a smart testing process (6033) to deploy tests on a testnet, deploy smart contract and verify source code, and a documentation module (6034) to document data and application products, AI model structure, parameter, performance metrics, and keep all the records. The data currency layer (603) interacts with the application layer (602) to operate and/or distribute, and/or allocate. The transaction and contract layer (604) provide the functions for data currency operation and transaction, comprising of a smart contract module (6041), a transaction management module (6042), a digital wallet (6043) to store and collect data currency, a satisfy factor authentication (6044) and a security keys maintenance (6045).

The transaction and contract layer (604) provide the operation and transaction functions for data currency layer (603). The security and regulation layer (605) comprising of security functions and provide regulation requirements for user layer (601), application layer (602), data currency layer (603), and transaction and contract layer (604). The security and regulation layer (605) provides an encryption and cyber security module (6051), which comprises an information security module (60511) for the central and individual data currency information security, an anti-theft and anti-piracy (60512) system for the management of data sources, and a digital oversight system (60513) to implement regulatory requirements by government agencies. The security and regulation layer (605) is used to enforce trading and transaction rules and regulatory requirement (6052) and monitor and maintain timing records of transaction (6053) for the transaction and contract layer (604). The security and regulation layer (605) provides a data currency intellectual property protection and disclosure system (5044) for the is data currency layer (603). The security and regulation layer (605) provides whites papers, laws, and regulations for user layer (601) and application layer (602).

### Definitions

FIG. 1 illustrates the system, interact and subsystem of data currency system for digital human healthcare and medical data exchange, analytics, and applications.
101: Model and application training module
102: Currency system
103: Data acquisition module
104: Data QA/QC and reporting module
105: Data currency owners and traders
106: Data currency trade and exchange platform
107: Human digital twin health and medical care platform
1011: Machine learning training
1012: Federated learning training
1021: World currency
1022: Cryptocurrency
1023: Data currency and valuation system
1024: Image data upload interface
1031: Hospital interface
1032: Personal interface
1033: Public agency interface
1034: Social interface
1041: Data revenue metric system
1042: Data cost metric system
1043: Data quality metric system
10231: Data feature-based valuation module
10232: Data currency generation and pooling
10233: Data currency unit lots
10234: Data currency allocation and distribution

FIG. 2 illustrates the procedure of data currency valuation factors.
201: Data Valuation Module
202: Value Deprecation
203: Value Appreciation
204: Data Currency Unit
2021: Increased Repeated and Outdated Dataset
2022: Increased Bad Records in Data Production
2023: Leaking, Counterfeit, and Illegal Usage of the Raw or Confidential Dataset
2024: Reduced Effective Data Usage
2025: Data Currency Trading Related Price Decrease
2031: Timeliness
2032: High-Frequency Effective Data Usage
2033: New Drug, Device/New Treatment
2034: Public Health Value
2035: Data Processing, Computing, and Production Cost
2036: Cyber Security Improvement
2037: Data-Assisted AI Model and Application Performance Improvement
2038: Data Currency Trading Related Price Increase

FIG. 3 illustrates the procedure of data currency volume system.
301: New data currency module
302: Parameter/Gradient update module
303: Hashing system
304: Data currency pooling system
305: Blockchain-enabled global ledger
306: Data currency assignment module
307: Calibration module
3011: New data calibration convergence
3012: New model calibration convergence
3013: New optimization method calibration convergence
3021: Track accuracy improvement
3022: Track convergence improvement
3023: Track performance metrics improvement
3031: Generate unique data currency lot ID
3041: Record/Store new data currency
3042: Record/Store status of new parameter/gradient combination
3043: Record/Store status of corresponding performance gain
3051: Register data currency generation
3052: Pooling each round update activity
3061: Distribute new data currency lots
3071: Calibration dataset oriented
3072: Evolution tree structure for large-scale parameter setting
3073: Form unique encryption keys/tokens FIG. 4 illustrates the factors of data currency contribution.
401: Federated and Machine Learning
402: Data Acquisition, Processing, and Labeling
403: Data Currency Exchange Platform
404: Valued Data Products
405: Data Currency Distribution
4011: Machine Learning/Deep learning assessment
4012: Federated Learning Assessment
4013: Machine Learning and Federated Learning Contributions
4014: Machine Learning- and Federated Learning-Based Data Currency Allocation
4021: Historical data Depreciation Module
4022: Dynamic and Real-time Data Valuation
4023: Rare and High-value Data Valuation
4031: Data Application Produce User Group Registration and Management
40131: Dataset Contributions
40141: Allocation Submodule
40142: Allocation Rule Integration Module
40211: Statistical Distribution Module for Decay
40221: Randomness of the Dataset
40222: Predictability of Dataset from Historical Data
40231: Targeted Data Types
40232: Number of Rare and High-value Data Occurrences/Frequencies
40311: Data Contributor who Upload Raw Data
40312: Data Collector, Surveyor, and Buyer
40313: Data Processor and Labeler
40314: Data Brokers and Reseller
40315: Data Application Developer and Operator
40316: Data Application End User
40317: Data Analyst
40318: Data User
401311: Increment in Gradients or Parameters changes
401312: Data Precision/Accuracy/Coverage Improvement
401411: Completeness and development of the model
401412: Processed or Used Percentage of Input and Label Data
401413: Improvement in Model Accuracy

FIG. 5 illustrates the trade platform and distribution of data currency.
501: Exchange function
502: Cryptocurrency transaction process function
503: Digital currency transactions logging and management system
504: Data application produce user groups registration and management system
505: Smart multi-party contracting
506: Security and user protection module
5011: Internal trading system
5012: External trading system
5013: Exchange rate mechanism
5021: Initiate transaction process
5022: Secure transaction function
5023: Point-to-point transaction
5024: Group-to-group transaction
5025: Validate process
5026: Certified process
5027: Recording process
5028: Transferring
5041: Raw data contributor
5042: Data collector, surveyors, buyers
5043: Data processor and labeler
5044: Data broker and reseller
5045: Data application developer/operator
5046: Data application end user
5047: Data analyst
5048: Data user through digital currency trader
50111: Data products using different data currency
50112: Difference data products using same data currency
50121: Other data currency
50122: Real world currency
50123: Cryptocurrency

FIG. 6 illustrates the operation and transaction system of data currency.
601: User layer
602: Application layer
603: Data currency layer
604: Transaction and contract layer
605: Security and regulation layer
6011: Initiate, approve and verify
6012: Initial price and smart contract offer
6013: Build and/or Maintain user community
6021: Application development interface with other digital currency
6022: Application development interface with trading and transaction
6023: Initial coin offering (ICO) and/or holding crowd sale
6024: Official release, transaction, operation and maintenance
6031: Blockchain platform
6032: Data currency token standard and template
6033: Smart testing process
6034: Documentation module
6041: Smart contracts module
6042: Transaction management module
6043: Digital wallet
6044: Satisfy factor authentication
6045: Security keys maintenance
6051: Encryption and cyber security module
6052: Enforce trading and transaction rules and regulatory requirement
6053: Monitor and maintain timing record of transaction
6054: Data currency intellectual property protection and disclosure system
6055: White papers, laws, and regulations
60511: Information security module
60512: Anti-theft and anti-piracy
60513: Digital oversight system

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The terms “memory,” “memory device,” “computer-readable storage medium,” “data store,” “data storage facility,” and the like each refer to a non-transitory device on which computer-readable data, programming instructions or both are stored. Except where specifically stated otherwise, the terms “memory,” “memory device,” “computer-readable storage medium,” “data store,” “data storage facility,” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions. Except where specifically stated otherwise, the singular term “processor” or “processing device” is intended to include both single-processing device embodiments and embodiments in which multiple processing devices together or collectively perform a process.

The terms “instructions” and “programs” may be used interchangeably herein. The instructions may be stored in object code format for direct processing by the processor, or in any other computing device language, including scripts or collections of independent source code modules that are interpreted on demand or compiled in advance. Functions, methods, and routines of the instructions are explained in more detail below. The instructions may be any set of instructions to be executed directly (such as machine code) or indirectly (such as scripts) by the processor. For example, the instructions may be stored as computing device code on the computing device-readable medium.

In addition, the terms “unit,” “-er,” “—or,” and “module” described in the specification mean units for processing at least one function and operation, and can be implemented by hardware components or software components and combinations thereof.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random is access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as

Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, is field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

Aspects of the present disclosure are described herein with reference to flowchart illustrations and/or block diagrams of methods, apparatus (systems), and computer program products according to embodiments of the invention. In some embodiments, the flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, is depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Unless specifically stated otherwise, it is appreciated that throughout the disclosure, descriptions utilizing terms such as “obtaining,” “performing,” “receiving,” “computing,” “associating,” “assigning,” “traversing,” “calculating,” “determining,” “identifying,” “transforming,” “ranking,” “providing,” “transmitting,” or the like, refer to the action and processes of a computer system, or similar electronic computing device, that manipulates and transforms data represented as physical (or electronic) quantities within the computer system memories or registers or other such information storage, transmission or display devices.

As used herein, the term “logistic regression” is a regression model for binary data from statistics where the logit of the probability that the dependent variable is equal to one is modeled as a linear function of the dependent variables.

As used herein, the term “neural network” is a machine learning model for classification or regression consisting of multiple layers of linear transformations followed by element-wise nonlinearities typically trained via stochastic gradient descent and back-propagation.

The term “machine learning,” as used herein, refers to a computer algorithm used to extract useful information from a database by building probabilistic models in an automated way.

The term “regression tree,” as used herein, refers to a decision tree that predicts values of continuous variables.

It will be understood that, although the terms “first,” “second,” etc., may be used herein to describe various elements, components, regions, layers and/or sections. These elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

It is noted here that, as used in this specification and the appended claims, the singular is forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention.

Further, the dates of publication provided may be different from the actual publication dates which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A medical and healthcare service platform, wherein the medical and healthcare service platform is supported by a digital data currency system and provides medical and healthcare data processing, analyzing, and predicting based on a digital human system by integrating participating parties comprising individual persons, researchers, healthcare providers, and regulatory and public sectors, wherein the medical and healthcare service platform comprising:
a digital human replica system that constructs a digital human replica to provide virtual representation, modeling, and visualization services based on present and past medical and health data of physical persons;
a digital human simuli system that constructs a digital human simuli to provide virtual simulation and modeling of future health and physiological evolution of a physical person based on the present and past medical and health data;
a digital human agent system that represents virtual medical and health service professionals with specialties and functions, wherein the virtual medical and healthcare service professionals are formed based on professional knowledge and capabilities, specialties, and experiences of physical medical and healthcare professionals and characteristics and specialties of non-medical and healthcare professionals or practitioners; and
a digital human data acquisition system that collects biometric identification and medical-related data,
wherein the digital data currency system awards data sharing and contributions in a full ecosystem of data generation comprising data processing, data cleaning and denoising, data encryption and anonymization, data labeling and calibration, and data analytics, data contributions related to medical and healthcare services; and services provided by medical and healthcare professionals from clinical practices, drug companies from laboratories or clinical trial data, and academic researchers from research work,
wherein the digital human replica system receives input data from the human data acquisition system for both a target physical person and other persons with similar biomedical, social-demographical, occupational, and lifestyle characteristics for building, calibrating, and customizing the digital human simuli system for the target physical person to simulate the growing and aging, disease events, injury events, and their reactions to medicines and treatment plans,
wherein the digital human agent system creates a special digital human replica of actions, treatment plans, and decision makings of medical and healthcare professionals, the digital human agent system configured to execute simulated medical, care, and health services as an intervention based on simulation in a digital human simuli model of the digital human simuli system,
wherein the digital human stimuli and the digital human agent system are integrated to perform model optimization to select and determine an optimal treatment or support plan to achieve an optimal health and medical outcome,
wherein the digital human digital data currency system awards digital currency for data contribution by the participating parties who interface with the digital human data acquisition system and wherein data contribution results in improvement of performance of the digital human simuli system and the digital human agent system, and
wherein the digital human digital data currency system comprises a data currency valuation subsystem that provides dynamic quotes of a digital currency based on values of corresponding data in the digital human digital data currency system.

2. The medical and healthcare service platform of claim 1, wherein the digital human digital data currency system further comprises:
i) a medical data input interface for data uploading and transferring from hospitals, individuals, public agencies, families, and communities,
ii) a data feature-based value evaluation subsystem that defines data currency valuation based on timeliness, acquisition or processing cost of data, frequency of data usage, new medical health advances including new artificial intelligence (AI) models, drugs, treatment, and medical devices, public and community health value increase, and data quality and security related dynamic features,
iii) a data quality assurance and quality control subsystem that ensures consistency and standards of data products,
iv) a data currency volume and ownership assignment subsystem that generates new data currency based on AI model performance improvement and distributes generated currency based on contributions of all participants of providing, processing, cleaning, and modeling data,
v) a data acquisition, processing, and labeling contribution assessment subsystem that conducts production of cleaned, anonymized, labeled datasets for healthcare AI models and applications,
vi) a hybrid federated and machine learning based contribution assessment system to assess contributions of data contributors with different direct data sharing or indirect AI model weights sharing for improving healthcare AI model and applications.

3. The medical and healthcare service platform of claim 2, wherein the medical data input interface is configured to collect medical related data and comprises:
a hospital interface for uploading and transferring of medical records from doctors and hospital data center,
a personal interface for individual to upload real-time or historical wearable healthcare device data, medical exam results, and direct text- or audio-based inputs,
a public agency interface to collect anonymous data collected from health checkpoint and population, and
a family and community interface for uploading case-by-case descriptions and audio and video data.

4. The medical and healthcare service platform of claim 1, wherein the data currency valuation subsystem uses data features for determining initial data value evaluation, the data features comprising: data types or categories for application; data stage of medicine trials; infectious disease severity designations; individual disease stage designation; data sources; social demographics; data fields; data coverage; data volume; or data resolution.

5. The medical and healthcare service platform of claim 1, wherein the data currency valuation subsystem determines monetary tokenized valuation for healthcare-related data, and wherein:
a) value appreciation is determined by one or more of the following factors:
timeliness of new or real-time data, wherein value of the data positively correlates with timeliness of the data;
high-frequency effective data usage;
a new drug, device, or treatment created or discovered;
a public health value added through contributions to new community and public health solutions to detect and control spreading and outbreak an infectious disease;
data processing, computing, and production cost in a full data production process;
cyber security improvement to enhance data security and improved counterfeit countermeasures;
data-assisted AI model and application performance improvement such as accuracy and coverage; and
data currency trading related price increase; or
b) value deprecation is determined by one or more of the following factors:
increased number of repeated or outdated datasets that do not provide new or useful information for healthcare model and application improvement;
increased number of bad records in data production comprising missing data, fake data, or biased data;
leaking, counterfeit, and illegal usage of raw or confidential datasets that lead to legal, technological, societal, and sale challenges of datasets in practices of healthcare model and application;
reduced effective data usage by models and applications; and
data currency trading related price decrease.

6. The medical and healthcare service platform of claim 1, wherein the data currency valuation subsystem determines value evaluation of medical-related data based on one or more of data features comprising:
a) standardization module of data production from raw data formatting, data processing procedure, analytic and labeling tools and functionality specifications, and data performance and quality assurance and control;
b) data quality assessment metrics comprising: comparison to validation and/or diagnostics datasets; detection or diagnostic accuracies; recovery rates and quality assessment; death, incidents, severe damage, injuries, or paralyzation rate;
c) erroneous and false data detection and correction methods comprising: sensor error and malfunctions; human input error or vagueness; or text and handwriting recognition error; and
d) multi-dataset cross-validation mechanisms comprising: spatial-temporal matching; correlation matching; or pattern matching between similar population groups.

7. The medical and healthcare service platform of claim 6, wherein the data quality assurance and quality control, and reporting subsystem comprises a periodic data quality reporting system that reports one or more of:
a) cost of data production and maintenance comprising one or more of: data generation and/or acquisition cost, data cleaning and labeling cost, data analytic cost, data storage cost, data application cost, and security, certification, or oversight cost;
b) number of applications and application revenue and monetization results in a reporting period comprising one or more of data application subscription and membership revenues; data application service revenues; data currency trading revenues; monetized values of derived products such as new drugs, treatment methods, derived data applications; and money savings for families, agencies, and communities compared with prior applications or methods; and
c) a data product quality metric system comprising one or more of:
i) output versus input instance ratios defined as a ratio between application service instances over number of input data instances in a reporting period;
ii) data growth capability defined by processed, labeled, and calibration-consumed data versus newly acquired or produced data rate based on size of training data set to achieve a latest model divided by a rate of new data acquisition through applications;
iii) incremental data benefit gains to keep track of different stages of data and application matureness, especially, monitoring mature datasets entering a long-tail stage where increased data consumption is needed to gain performance improvement of trained AI models and applications;
iv) user growth potentials based on a growth rate of users of healthcare products or applications produced by the data, average cost per user data acquisition and production, and usage activity data based on active user engagement time and interactions; and
v) data supported models, publications, and application increasing rates.

8. The medical and healthcare service platform of claim 1, wherein the data currency volume and ownership assignment system allocates and distributes currency based on each round of optimization of parameters and gradients of the AI model, and wherein the data currency volume and ownership assignment system comprises one or more of:
a) a new data currency module that is generated with every round of calibration converges triggered by new data, new model, new optimization method;
b) a parameter or gradient update module to keep track of performance improvement in accuracy, coverage, and other performance metrics of the data;
c) a hashing system that generate unique data currency lot ID based with creating a hash code corresponding to updated model performance metrics, parameters, or gradients;
d) a data currency pooling system to record and store new data currency lots and status of a new gradient or parameter combination and corresponding performance gains;
e) a blockchain-enabled global ledger to register data currency generation and pooling activities with every round of such updates; and
f) a data currency assignment module that distributes new data currency lots in a new currency pool to all contributors of calibration or training, including new data contributors to raw, labeled, and ground truth data, computing resource providers for edge or cloud computing depending on AI technology architectures, validators, data and file transmission, or cyber security measures.

9. The medical and healthcare service platform of claim 8, wherein the data currency valuation subsystem comprises a calibration data currency volume and ownership assignment system comprising one or more of: calibration datasets oriented; large-scale parameter settings that use a tree structure to compress and tokenization; and all sets of parameters to be created in a way that follows an evolution tree; and new currency key or token with encryption essentially mapping uniqueness of structure and uniqueness of data nodes to form a unique encryption key or token.

10. The medical and healthcare service platform of claim 8, wherein the data acquisition, processing, and labeling contribution assessment system comprises one or more of:
a) historical data depreciation methods by using statistical distribution models for modeling and calibrating decay with respect to time;
b) dynamic and real-time data valuation methods for determining randomness of datasets or predictability of the datasets from historical data; and
c) rare and high-value data valuation metrics comprising targeted data types, number of rare, high-value data occurrences, or frequencies.

11. The medical and healthcare service platform of claim 1, wherein the data currency valuation subsystem comprises a federated and machine learning contribution assessment system comprising one or more of:
i) direct machine learning or deep learning assessment where currency volume is allocated based on performance improvement of models and applications through centralized machine learning model calibration with direct raw data and data labels produced at a data center;
ii) a federated learning assessment module that assesses currency volume only based on performance improvement and parameter, weight or gradient updates from federated learning process where raw data are not shared with a data center;
iii) machine learning and federated learning contributions comprising: dataset contributions with respect to increment in gradients or parameters changes or dataset contributions in improvement of data precision, accuracy, or coverage;
iv) machine-learning- and federated-learning-based data currency allocation based on one or more of:
a) an allocation submodule based on completeness and development of a model where allocation will increase with higher average change rates of gradient or parameters in an AI model as a result of a training process, and will decrease if average change in parameters or gradients of the model caused by the dataset used is not significant;
b) an allocation submodule based on processed or used percentage of input and label data;
c) an allocation submodule based on improvement in model accuracy where allocation increases with larger accuracy improvement beyond a pre-defined no-change threshold specified by a data product and decreases with smaller accuracy improvement in trained models; and
d) an allocation rule integration module that determines final allocation based on a combined assessment of the submodules set forth in a)-c) based on characteristics of data products.

12. The medical and healthcare service platform of claim 1, wherein the data currency valuation subsystem comprises a data currency exchange platform comprising one or more of the following functionalities:
a) data application that produces user groups registration and management including one or more of: data contributors who upload raw data; data collectors, surveyors, and buyers; data processors and labelers; data brokers and resellers; data application developers or operators; data application end users; data analysts that produce insights towards developing corresponding models and applications; and data users through digital currency traders and transactions;
b) a data currency exchange cryptocurrency transaction process function comprising one or more of: initiate transaction process; secure transaction function; point-to-point transaction; transaction group-to-group; validate process; certify process; recording process;
and transferring;
c) smart multi-party contracting among data contributors;
d) digital currency transactions logging and management system;
e) currency token circulation within an exchange platform;
f) toke circulation and exchange with data currency from other exchange platforms or other currency or cryptocurrencies; and
g) security and user protection modules.

13. The medical and healthcare service platform of claim 12, wherein the data currency exchange platform has a data currency exchange function for internal platform circulation,
wherein within one data currency platform, data currency from different data currencies can be tradeable to be used to purchase or exchange data products under other data currencies,
wherein different types of data products are purchased, exchanged, and acquired, using the same data currency,
wherein for external exchange with other currencies, the data currency platform comprises an exchange module for data currencies from other data currency exchange systems, an exchange module with world currency with a specific exchange rate, or an exchange module with other cryptocurrencies, and
wherein exchange rates are determined by one or more of characteristics: an exchange rate system between different data currency types or similar data products under different data currency; exchange with world currency once money values are determined through initial valuation process; all data products have value, but not all data products have transaction value; some data products are protected and never tradeable; and a need to define a method for dividing data products.

14. The medical and healthcare service platform of claim 1, wherein the data currency valuation subsystem performs a data currency global certification process that allows distributed systems to work together and stay secure for generating data currency, the data currency global certification process comprising one or more of:
a) Proof-of-Work (PoW) consensus mechanisms based on hours or result instances processed, analyzed, labeled, or computed by different data contributors; and
b) Proof-of-Stake (PoS) consensus mechanisms based on data currency generation and allocation module outcomes.

15. The medical and healthcare service platform of claim 1, wherein the data currency operation and transaction subsystem comprises a data currency development framework for building, distribution, issuance, regular disclosure, property rights protection, the data currency development framework comprising one or more of:
a) encryption and cyber security modules that comprises central and individual data currency information security modules; anti-theft and anti-piracy management for data sources; and digital oversight system to implement regulatory requirements by government agencies;
b) data currency blockchain platform to provide global verifiable ledgers to document data currency transactions and activities;
c) data currency token or coin development modules;
d) data currency smart testing process comprising: deploying tokens on a testnet;
deploying smart contracts; or verifying source code;
e) data currency smart contracts and wallet modules for individual data currency keeping and exchange, which performs one or more operations of: (i) transaction management of smart contracts; (ii) initiating, approving, and verifying transfers or trades; (iii) satisfying factor authentications and maintenance security keys; (iv) enforcing trading and transaction rules and regulatory requirements; (v) initial price and smart contract offering; (vi) monitoring and maintaining timing records of transactions; and
f) data currency intellectual property protection and disclosure systems.

16. The medical and healthcare service platform of claim 1, wherein the data currency development framework comprises a data currency token or coins development platform comprising one or more of:
a) data currency tokens standards and templates;
b) data currency digital wallet;
c) smart contract interface;
d) transfer function interface;
e) record keeping interface to document name, symbol, and decimal of a token;
f) initial total supply management interface;
g) data and application product documentation interface;
h) AI model structure, parameter, and performance metrics documentation interface;
i) programming interface and coding environment;
j) application development interfaces to interact with other digital currencies; and
k) application development interfaces to interact with trading and transaction systems.

17. The medical and healthcare service platform of claim 1, wherein the data currency development framework comprises a data currency smart distribution process comprising one or more of: initial coin offering (ICO) or holding a crowd sale; build or maintain user community; white papers, laws, and regulations; and official release, transaction, operation, and maintenance.
